(19) United States
(12) Patent Application Publication
(10) Pub. No.: US 2008/0014281 A1
(43) Pub. Date: Jan. 17, 2008

(54) Chitin Micro-Particles As An Adjuvant

(75) Inventors: Yoshimi Shibata, Boca Raton, FL (US); Quentin N. Myrvik, Caswell Beach, NC (US)

Correspondence Address: AKERMAN SENTERFITT, P.O. BOX 3188, WEST PALM BEACH, FL, 33402-3188, US

(73) Assignee: FLORIDA ATLANTIC UNIVERSITY, Boca Raton, FL (US)

(21) Appl. No.: 11/763,941

(22) Filed: Jun. 15, 2007

Related U.S. Application Data
(60) Provisional application No. 60/814,382, filed on Jun. 16, 2006.

(51) Int. Cl. A61K 31/722; A61K 39/04; A61K 9/14; A61P 29/00; A61P 37/02; C07H 15/00

(52) U.S. Cl. 424/499; 424/248.1; 514/25; 536/17.2

(57) ABSTRACT

A composition and method for the preparation of micro-particles of chitin (a naturally occurring polymer of N-acetyl-D-glucosamine), the characterization of chitin micro-particles as an immune adjuvant and the use of chitin micro-particles to enhance protective immunity against intracellular infectious agents and diseases as well as to inhibit allergic responses and diseases.

### CROSS-REFERENCE TO RELATED APPLICATIONS

The present application claims the priority of U.S. Provisional Patent Application No. 60/814,382, filed Jun. 16, 2006, entitled “CHITIN MICRO-PARTICLES AS AN ADJUVANT” which is incorporated herein by reference in its entirety.

### STATEMENT REGARDING FEDERALLY SPONSORED RESEARCH OR DEVELOPMENT

The United States Government may have certain rights in this invention pursuant to the National Institute of Health Grant No. 5 RO1 HL-071711-04 and the United States Army Grant No. DAMD17-03-01-0004.

### FIELD OF THE INVENTION

The invention relates to the field use of T cell adjuvants in animals and humans for the prevention and treatment of infectious and immunologic diseases.

### BACKGROUND

Certain microorganisms could threaten health of all populations, especially immunocompromised (elderly, ill, pregnant women, and young children). Intake of organisms can result in life-threatening infectious diseases. In addition, environmental allergens cause a variety of immediate hypersensitivity diseases including asthma. A practical immunomodulator, a Th1 adjuvant, that could stimulate host defense mechanisms would represent a general approach to promotion of host defense against intracellular pathogens including multi-drug resistant bacteria as well as against immediate hypersensitivity diseases. However, there is no clinically appropriate Th1 adjuvant currently available for this application.

### SUMMARY

The invention relates to the preparation of micro-particles of chitin (a naturally occurring polymer of N-acetyl-D-glucosamine), the characterization of chitin micro-particles as an immune adjuvant and the use of chitin micro-particles to enhance protective immunity against infectious agents such as, Listeria monocytogenes, an infectious agent causing food poisonings and miscarriage of fetus, in susceptible animal models.

In a preferred embodiment, an adjuvant composition comprising at least one chitin micro-particle wherein the micro-particles are about 0.01 μm up to 20 μm in diameter.

In another preferred embodiment, the chitin micro-particles are between about 1 μm to 10 μm in diameter.

In another preferred embodiment, the chitin micro-particles are between about 1 μm to 4 μm.

In a preferred embodiment, the chitin micro-particles are suspended in a pharmaceutical composition in an amount effective in decreasing T-helper type 2 cell activity and increase T-helper type 1 cell activity. Preferably, the chitin micro-particles are present in a concentration of about 1×10³ particles/mg up to 5×10⁹ particles/mg. Also preferred, are concentrations of about 1×10⁸ particles/mg to about 6×10⁸ particles/mg.

In another preferred embodiment, the chitin micro-particles are present at a concentration of about 1×10⁸ particles/mg to about 3×10⁸ particles/mg when the chitin micro-particle diameter is about 1 μm to about 10 μm. Also preferred, are concentrations of chitin micro particles of about 3×10⁸ particles/mg to about 6×10⁸ particles/mg when the chitin micro-particle diameter is about 1 μm to about 4 μm.

In another preferred embodiment, the composition further comprises Mycobacterial antigens, MDP-59, ragweed allergens, peanut allergens, tree nut allergens, pollen allergens, house dust mite antigens, cockroach allergens, ovalbumin, mycobacterial heat-shock protein 65, antigens, and purified protein derivative (PPD) and derivatives thereof.

In another preferred embodiment, the chitin micro-particles can be administered as a composition comprising vaccines, tumor antigens, viral antigens, bacterial antigens, or peptides. The chitin micro-particles can be administered prior to, in conjunction with or during and after administration of a vaccine.

In another preferred embodiment, a method of preparing a chitin micro-particle comprises washing chitin crude sources with endotoxin free saline; mixing the washed chitin with acid; extracting and isolating acid soluble chitin; neutralizing and precipitating soluble chitin; washing water-insoluble chitin; lyophilizing water-insoluble chitin; re-suspending water-insoluble chitin in endotoxin free saline; filtrating chitin particles through micro-pores; preparing a 1-10 μm chitin micro-particle. In one aspect the chitin micro-particles are about 1 to 4 μm. The prepared chitin micro-particles can be stored long term in saline are stable for at least one year at 4° C.

In another preferred embodiment, a method of treating and/or preventing an inflammatory disease comprises administering to a patient a therapeutically effective dose of chitin micro-particles wherein the chitin micro-particles are between about 1 μm to 4 μm.

In another preferred embodiment, the chitin micro-particles are administered at an amount comprising about 0.1-500 mg per kg of the patient.

In another preferred embodiment, the chitin micro-particles are administered at an amount comprising about 0.1-10 mg per kg of the patient intraperitoneally or subcutaneously.

In another preferred embodiment, the chitin micro-particles are administered at an amount comprising about 0.1-1 g per kg of the patient when administered orally.

In another preferred embodiment, the chitin micro-particles are suspended in a pharmaceutical composition in an amount effective in decreasing T-helper type 2 cell activity and increase T-helper type 1 cell activity. The route of administration can be oral, intra-peritoneal, intra-venous and/or under a patient's skin.

In another preferred embodiment, administration of chitin micro-particles is therapeutically effective in down-regulating Th2-mediated diseases including allergic asthma, food allergy and allergic dermatitis.

In another preferred embodiment, administration of chitin micro-particles is therapeutically effective in up-regulating Th1-host defenses against intracellular infections.

In another preferred embodiment, a method of modulating an immune response, comprises administering to an animal an effective dose of chitin particles wherein the chitin micro-particles are between about 1 μm to 4 μm in diameter.

In another preferred embodiment, the chitin micro-particles are suspended in a pharmaceutical composition in an amount effective in decreasing T-helper type 2 cell activity and increase T-helper type 1 cell activity.

In another preferred embodiment, the route of administration is oral, intra-peritoneal, intra-venous and/or under a patient's skin.

In some embodiments the concentration of chitin particles are about 1×10³ to about 5×10⁸ particles/mg for 1-10 μm chitin; and 1×10⁴ to about 1×10⁹ particles/mg for 1-4 μm chitin.

Other aspects are described infra.

### BRIEF DESCRIPTION OF THE DRAWINGS

The invention is pointed out with particularity in the appended claims. The above and further advantages of this invention may be better understood by referring to the following descriptions in conjunction with the accompanying drawings, in which:

FIG. 1 is a schematic representation illustrating the mechanisms of action of host defense mechanisms when chitin micro-particles are given to hosts.

FIG. 2 is a schematic representation illustrating the mechanisms in the use of treating mammalian disorders.

FIG. 3 is a graph showing that the chitin particles induced TNFα producing activities at levels comparable to those by bacterial CpG and LPS.

FIGS. 4A and 4B are graphs showing intraperitoneal (IP) administration of chitin particles enhances reactive nitrogen and oxygen intermediates release by peritoneal macrophages (FIG. 4A) and splenic macrophages (FIG. 4B). The results show that these macrophages released superoxide anion (ROS) as well as nitric oxide (NO, RNS). C57Bl/6 mice received 1 mg chitin micro-particles ip. At indicated intervals, peritoneal and splenic macrophages were isolated. To measure NO release by Griess Reagent, macrophages were stimulated with LPS (1 μg/ml) for 24 hrs. To measure superoxide anion release by cytochrome c reduction, macrophages were stimulated with PMA (1 μM) for 1 hr. Mean±SD, n=3.

FIGS. 5A and 5B are graphs showing that chitin micro-particles induce TNFα (FIG. 5B) but not detectable IL-10 (FIG. 5A) production in RAW264.7 macrophages. RAW264.7 cells (10⁶ cells/ml) were stimulated with chitin micro-particles (100 μg/ml), CpG-ODN (5 μg/ml), GpC-ODN (5 μg/ml), heat-killed Mycobacterium bovis BCG (HK-BCG) (100 μg/ml), LPS (1 μg/ml), soluble chitin oligosaccharide (1 mg/ml), >50 μm chitin particles (1 mg/ml), 1-10 μm chitosan particles (1 mg/ml) or 1.1 μm latex beads (1 mg/ml) at 37° C. for 3, 6 and 24 hrs. The levels of IL-10 and TNFα were detected by specific ELISAs. Mean±SD, n=4. *p<0.05; #p<0.01, compared to cells treated with saline at the same time point.

FIGS. 6A and 6B are graphs showing that chitin micro-particles induce TNFα (FIG. 6B), but not detectable IL-10 (FIG. 6A) production in mouse spleen macrophages. Mouse spleen macrophages were isolated from normal C57Bl/6 mice. Spleen cells (2×10⁶ cells/ml) were stimulated with 1-10 μm chitin particles (100 μg/ml), CpG-ODN (5 μg/ml), GpC-ODN (5 μg/ml), heat-killed Mycobacterium bovis BCG (HK-BCG) (100 μg/ml), LPS (1 μg/ml), soluble chitin oligosaccharide (1 mg/ml), >50 μm chitin particles (1 mg/ml), 1-10 μm chitosan particles (1 mg/ml) or 1.1 μm latex beads (1 mg/ml) at 37° C. for 3 hrs for TNFα production and 24 hrs for IL-10 production. The levels of TNα and IL-10 were detected by specific ELISAs. Mean±SD, n=4. *p<0.05; #p<0.01, compared to cells treated with saline at the same time point.

FIG. 7 is a scan of Western blots showing that chitin micro-particles, CpG-ODN, HK-BCG and LPS activate p38, ERK1/2, and JNK in RAW 264.7 cells. Cells (10⁶ cells/ml) were stimulated with chitin micro-particles (100 μg/ml), CpG-ODN (5 μg/ml), GpC-ODN (5 μg/ml), HK-BCG (100 μg/ml), LPS (1 μg/ml), soluble chitin oligosaccharide (1 mg/ml), >50 μm chitin particles (1 mg/ml), chitosan micro-particles (1 mg/ml) or 1.1 μm latex beads (1 mg/ml) at 37° C. for 0, 10, 20, 30, and 40 min. Isolated macrophage proteins were separated on 11% SDS-polyacrylamide gel and electroblotted to PVDF membrane. Phosphorylated MAPK (P-p38, P-Erk1/2 and P-JNK) were detected with specific antibodies.

FIG. 8 is a graph showing the effects of chitin treatment on serum IgE levels in ragweed-sensitized Balb/c mice. Groups of mice (7/group) were given chitin (8 mg/mouse/day) orally 3 days before ragweed immunization and continued to receive chitin during the immunization periods. Immunized mice receiving saline (0.5 ml/mouse/day) served as controls. Blood samples were collected from tail veins at indicated days. Total IgE levels in the blood samples were measured by ELISA. Mean±SD, n=7, **, p<0.01; #, p<0.0005 compared with the saline-treated group.

FIG. 9 is a graph showing development of MPB-59-induced footpad delayed type hypersensitivity in C57Bl/6 (WT) and IL-10-knockout (IL-10-KO) mice co-immunized with MPB-59 and chitin micro-particles. WT (white bars) and IL-10 KO (black bars) mice were immunized with MPB-59, MPB-59/chitin, chitin and saline as indicated. Seven days after the final immunization, WT and IL-10 KO mice received 50 μg of MPB-59 solution in right footpad and 50 μl of saline in left footpad (control). After 48 hours, right footpad thickness minus left footpad thickness in each group of mice was obtained. Mean±SD, n=6.

FIG. 10 is a graph showing the capacity of PGE₂ biosynthesis that is down-regulated by administration of chitin micro-particles. C57Bl/6 mice received chitin particles (1 mg) intraperitoneally. Peritoneal macrophages were isolated 24 hrs after the stimulation. Macrophage suspensions (10⁶/ml) were stimulated with 1 μM calcium ionophore 23187 (black bars) or medium (gray bars), for 2 hrs to determine PGE₂ release. PGE₂ was assayed by ELISA. Mean±SD, n=3.

FIG. 11 is a graph showing results obtained with the new 1-4 μm chitin preparations. 1-10 μm chitin particles that were prepared as described in the Examples section were filtered through meshes and differential centrifugation. Three fractions were obtained with fine (1-4 μm), medium (4-7 μm) and coarse (7 - 10 Jim) particles. Sizing was cytometrically measured. These fractioned particles (20 μg/ml) were added to the macrophage and NK cell-containing spleen cell cultures as described above. After 24 hr incubation, IL-12 (black bars) and IFNγ (white bars) levels in the supernatants were measured by ELISA. Mean±SD, n=3−4.

FIG. 12 are blots showing the effects of chitin particles on the levels of CpG-ODN-induced IL-10 mRNA.

### DETAILED DESCRIPTION

A composition comprising micro-particles of chitin. Methods of the preparation of micro-particles of chitin (a naturally occurring polymer of N-acetyl-D-glucosamine), the characterization of chitin micro-particles as an immune adjuvant and the use of chitin micro-particles to enhance protective immunity against infectious agents.

Definitions

As used herein, “subject” or “patient” refers to the recipient of the therapy to be practiced according to the invention. The subject can be any vertebrate, but will preferably be a mammal. If a mammal, the subject will preferably be a human, but may also be a domestic livestock, laboratory subject or pet animal.

As used herein, “substance” refers to any substance to which an immune response may be directed, and includes antigens and pathogens.

As used herein, “exposure” to a substance, e.g. antigen includes both natural, environmental exposure to the substance as well as administration of the substance to a subject.

The term “vaccine composition” intends any pharmaceutical composition containing an antigen, which composition can be used to prevent or treat a disease or condition in a subject. Vaccine compositions may also contain one or more adjuvants. Typically a vaccine composition is used for the prophylaxis of a disease caused by a pathogen, however, the vaccine compositions of the present invention can also be used in a therapeutic context.

An “immunological response” or “immune response” against a selected agent, antigen or a composition of interest is the development in an individual of a humoral and/or a cellular immune response to molecules (e.g., antigen) present in the agent or composition of interest. For purposes of the present invention, a “humoral immune response” refers to an immune response mediated by antibody molecules, while a “cellular immune response” is one mediated by T-lymphocytes macrophages and/or other white blood cells. Mammalian immune responses are understood to involve an immune cascade following one of two broad categories of response, characterized by the class of T helper cell which initiates the cascade. Thus, an immune response to a specific antigen may be characterized as a T helper 1 (Th1)-type or T helper 2 (Th2)-type response, depending on the types of cytokines that are released from antigen-specific T lymphocytes following antigen presentation. Th1 immune responses are generally characterized by the release of inflammatory cytokines, such as IL-2, interferon-gamma (IFN-γ), and tumor necrosis factor alpha (TNF-α), from the antigen-stimulated T helper cells. Th1 responses are also associated with strong cellular immunity (e.g., CTLs) and the production of IgG antibody subclasses that possess opsonizing and complement-fixing activity, such as IgG2a in the commonly used mouse model. On the other hand, Th2 immune responses are characterized by the release of noninflammatory cytokines, such as IL-4 and IL-10, following stimulation of antigen-specific T helper cells. The Th2 responses generally do not favor maximal CTL activity, but are associated with strong antibody responses, representing IgG subclasses such as IgG1 in the mouse, antibody classes that lack opsonizing and complement-fixing activity. In general, the antibody levels associated with Th2 responses are considerably stronger than those associated with Th1 responses.

The term “adjuvant” intends any material or composition capable of specifically or non-specifically altering, enhancing, directing, redirecting, potentiating or initiating an antigen-specific immune response. Thus, coadministration of an adjuvant and an antigen (e.g., as a vaccine composition) may result in a lower dose or fewer doses of antigen being necessary to achieve a desired immune response in the subject to which the antigen is administered. In certain embodiments of the invention, coadministration of an adjuvant with an antigen can redirect the immune response against the antigen, for example, where the immune response is redirected from a Th2-type to a Th1-type immune response, or vice versa. The effectiveness of an adjuvant can be determined by administering the adjuvant with a vaccine composition and vaccine composition controls to animals and comparing antibody titers and/or cellular-mediated immunity against the two using standard assays such as radioimmunoassay, ELISAs, CTL assays, and the like, well known in the art. Typically, in a vaccine composition, the adjuvant is a separate moiety from the antigen, although a single molecule can have both adjuvant and antigen properties (e.g., cholera toxin). For the purposes of the present invention, an adjuvant is used to either enhance the immune response to a specific antigen, e.g., when an adjuvant is coadministered with a vaccine composition, the resulting immune response is greater than the immune response elicited by an equivalent amount of the vaccine composition administered without the adjuvant, or the adjuvant is used to redirect or reverse the nature of the immune response from a Th2 to a Th1 response. In addition, for the purposes of the present invention, an “effective amount” of an adjuvant will be that amount which enhances an immunological response either alone, i.e. activates the Th1 response or redirects the response from a Th2 to a Th1 response, or an “effective amount” of an adjuvant will be that amount which is sufficient to bring about a shift or redirection of the immune response relative to the immune response to the antigen alone.

An “adjuvant composition” intends any pharmaceutical composition containing the chitin micro-particles of the invention.

As used herein, enhancing “innate immunity” includes enhancing activation of macrophages, NK cells, antigen presenting cells (APCs), and other elements known to be involved in immediate protection against subsequent exposure to a wide variety of microbial pathogens. Enhancement of innate immunity can be determined using conventional assays for activation of these elements, including but not limited to assays described in the examples set forth below.

As used herein, “enhancing a Th1 immune response” or “modulating an immune response” in a subject is evidenced by: (1) a reduction in levels of IL-4 or IL-5 measured before and after antigen challenge; or detection of lower (or even absent) levels of IL-4 in a treated subject as compared to an antigen-primed, or primed and challenged, control; (2) an increase in levels of IL-12, IL-18 and/or IFN (α, β, or γ) before and after antigen challenge; or detection of higher levels of IL-12, IL-18 and/or IFN (α, β, or γ) in a subject treated with the chitin comprising compositions as compared to an antigen-primed or, primed and challenged, control; (3) production of IgG2a antibody or its human analog in a treated subject; (4) a reduction in levels of antigen-specific IgE as measured before and after antigen challenge; or detection of lower (or even absent) levels of antigen-specific IgE in a subject treated with the chitin comprising compositions as compared to an antigen-primed, or primed and challenged, control; and/or (5) induction of a cytotoxic T lymphocyte (“CTL”) response in a treated subject.

Chitin Compositions

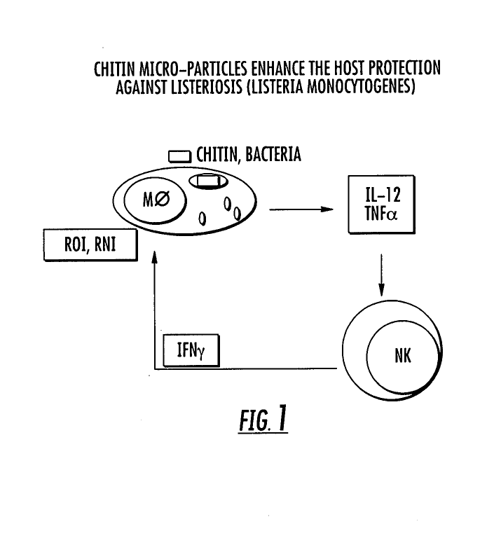

When chitin micro-particles are given to hosts (experimental animals), the host defense mechanisms are promoted as indicated in FIG. 1. Macrophages (MØ) phagocytose (uptake) chitin micro-particles in a mechanism similar to macrophages phagocytosing infectious bacteria. Macrophages produce Th1 cytokines including IL-12 and tumor necrosis factor-alpha (TNFα) within 3 hrs to 24 hours. These Th1 cytokines activate natural killer (NK) cells that will produce interferon-gamma (IFNγ) within 12-24 hours. IFNγ activates macrophages which generate reactive oxygen and nitrogen intermediates (ROI and RN) including superoxide anion and nitric oxide within 1-3 days after chitin micro-particle administration.

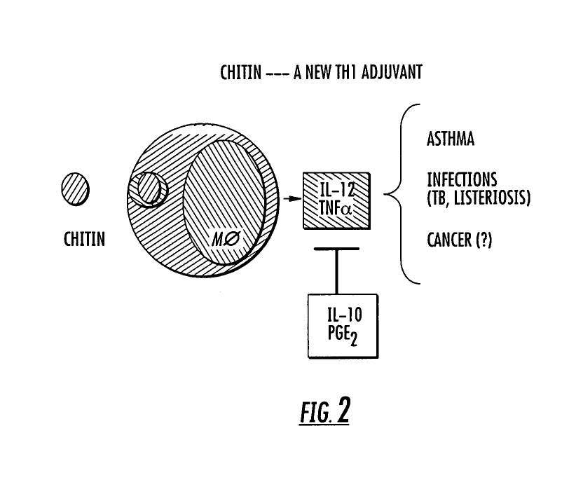

As shown in FIG. 2, since Th1 cytokines including IL-12 and TNFα modify host immune responses, chitin micro-particles are useful for therapy against asthma, infections (listeriosis, tuberculosis [TB]), and cancers. Furthermore, unlike other Th1 adjuvants that are prepared from bacterial components, chitin micro-particles do not induce IL-10 and prostaglandin E₂ (PGE₂). Both IL-10 and PGE₂ are known to inhibit Th1 adjuvant activities indicated in FIGS. 1 and 2. Thus chitin is the most potent Th1 adjuvant presently available and is an attractive candidate for stimulation of biodefense in immunocompromised populations where host defenses are down-regulated by increased levels of PGE₂ and IL-10.

In a preferred embodiment, the invention provides a method for enhancing an immune response to a substance, such as an antigen administered to a subject, or a pathogen to which the subject is exposed. The method can be used to modulate the magnitude, the duration, and the nature of the immune response to subsequent exposure to an antigen. The method comprises administering a composition comprising chitin micro-particles that can be administered to a subject either alone or prior to, in conjunction with or after administration of a vaccine or other therapeutically effective molecule. This “priming” of the subject with chitin micro-particles of the invention prior to antigen administration or pathogen exposure results in amplification of the Th1 immune response. Priming with the chitin compositions also shifts the nature of the immune response from a Th2 type response to a Th1 type response.

Examples of an immune response that can be enhanced by the method of the invention include, but are not limited to, activation of innate immunity (e.g., macrophages, natural killer (NK) cells), a Th1 response, and a cytotoxic T lymphocyte (CTL) response. The method can be used prophylactically or therapeutically.

Because priming activates innate immunity, the method of the invention can be used to protect against subsequent infection by a pathogen, such as a viral, bacterial, parasitic or other infectious agent. Preferably, the substance is a pathogen or an antigen associated with an infectious disease, an allergen or a cancer. Examples of infectious diseases include, but are not limited to, viral, bacterial, mycobacterial and parasitic diseases.

Certain pathogens, as well as certain cancers, are effectively contained by an immune attack directed by CD4⁺ T cells, known as cell-mediated immunity. Other pathogens, such as poliovirus, also require antibodies, produced by B cells, for containment. These different classes of immune attack (T cell or B cell) are controlled by different subpopulations of CD4⁺ T cells, commonly referred to as T-helper type 1 cells (Th1) and T-helper type 2 (Th2) cells.

The two types of T-helper (Th) cell subsets have been well characterized in a murine model and are defined by the cytokines they release upon activation. The Th1 subset secretes IL-2, IFN-γ and tumor necrosis factor, and mediates macrophage activation and delayed-type hypersensitivity response. The Th2 subset releases IL-4, IL-5, IL-6 and IL-10, which stimulate B cell activation. The Th1 and Th2 subsets are mutually inhibiting. For example, IL-4 inhibits Th1-type responses, and IFN-γ inhibits Th2-type responses. Similar Th1 and Th2 subsets have been found in humans, with release of cytokines identical to those observed in the murine model. Amplification of Th2-type immune responses is central to protecting against metazoan parasites, e.g. Schistosoma. In addition, a Th2-type response is important in the induction and maintenance of allograft tolerance and the maintenance of successful pregnancy. In contrast, suppression of a Th2-type response and amplification of a Th1-type immune response is of key importance in the treatment of diseases including cancers and disorders of the respiratory system, such as tuberculosis, sarcoidosis, asthma, allergic rhinitis and lung cancers.

Asthma is a common disease, with a high prevalence in the developed world. Asthma is characterized by increased responsiveness of the tracheobronchial tree to a variety of stimuli, the primary physiological disturbance being reversible airflow limitation, which may be spontaneous or drug-related, and the pathological hallmark being inflammation of the airways. The immune response producing airway inflammation in asthma is brought about by the Th2 class of T cells which secrete IL-4, IL-5 and IL-10. It has been shown that lymphocytes from the lungs of atopic asthmatic patients produce IL-4 and L-5 when activated. Both IL-4 and IL-5 are cytokines of the Th2 class and are required for the production of IgE and involvement of eosinophils in asthma. Thus reversal of a Th2 response and enhancement of a Th1 response is highly beneficial in the treatment of asthma.

Another disorder with a similar immune abnormality to asthma is allergic rhinitis. Allergic rhinitis is a common disorder and is estimated to affect at least 10% of the population. Allergic rhinitis may be seasonal (hay fever) and caused by allergy to pollen. Non-seasonal (perennial) rhinitis is caused by allergy to antigens such as those from house dust mite or animal dander. The abnormal immune response in allergic rhinitis is characterized by the excess production of IgE antibodies specific against the allergen. The inflammatory response occurs in the nasal mucosa rather than further down the airways as in asthma. Like asthma, local eosinophilia in the affected tissues is a major feature of allergic rhinitis. As with asthma, the reversal of a Th2 immune response and enhancement of a Th1 response is central to successful treatment.

Most food-allergic responses involve food-specific IgE-mediated reactions. IgE antibodies and allergens activate mast cells and basophils through the high-affinity IgE receptor. This activation causes the release of histamine and other mediators, leading to systemic anaphylactic reactions. As with asthma, the reversal of a Th2-mediated IgE formation is essential to successful treatment. Thus, in a preferred embodiment, treating a patient suffering from or at risk of a food allergy comprise administering to the patient an amount of the chitin micro-particles effective to treat the food allergy. This treatment results in the reversal of the Th2 response and subsequent events involved in allergies. As with any of the treatments and methods of treatment described herein, the chitin micro-particles can be administered orally in the form of a food supplement, a tablet, in suspension and the like. For example, as a dietary supplement, the adjuvants are mixed with any foods/drinks that are at around neutral to lower pH (2-7) and isotonic or mouth washer/tooth paste. Since the adjuvant is relatively heat-stable (autoclavable), the adjuvant can be added to any cooked/heated dishes (vegetable, rice, fish, and meat), fruit juice, milk, coffees and teas.

In another preferred embodiment, the invention provides a method for enhancing an immune response. The method can be used to modulate the magnitude, the duration and/or the quality of the immune response to a subsequently administered antigen or to subsequent exposure to a substance such as a pathogen.

In a preferred embodiment, the method enhances the Th1 response. The chitin micro-particles function as an immune shift adjuvant. An “immune shift adjuvant” is an adjuvant that is effective to alter or direct (re-direct) the nature of an immune response. The altering or redirecting is relative to the nature of the immune response that is directed against the antigen in the absence of the immune shift adjuvant. Thus, such chitin micro-particles are used herein to shift the nature of an immune response elicited against a selected antigen to favor a Th1-type response in lieu of a Th2-type response. The ability of an adjuvant to serve as an immune shift adjuvant can be determined by assessing the nature of immune responses engendered by, for example, administration of the vaccine composition alone, and administration of the vaccine composition with the adjuvant. This assessment can involve a characterization or identification of the types of cytokines that are released from antigen-specific T lymphocytes following antigen presentation in an individual and/or the characterization or identification of the predominate IgG subclasses that are elicited by an antigen/adjuvant combination relative to antigen alone. All of these characterization or identifications are well within the skill of the ordinarily skilled artisan as directed by the present specification. Specific methods are detailed in the Examples section which follows.

In another preferred embodiment, the method enhances or shifts the production of antibodies that recognize the substance. Changes in antibody production can be determined by detecting increased antibody levels in a subject or subjects pre-primed with the chitin compositions as compared to antibody levels in a subject or subject not receiving the chitin comprising composition prior to antigen administration. Enhanced antibody production can also include increasing the production of one class of antibody relative to production of another, less desirable class of antibody. For example, production of IgG2a antibodies can be enhanced while levels of IgE antibodies are reduced.

The immune response can also be enhanced by shifting the response from a Th2 to a Th1 type response. As used herein, “Th1/Th2 response(s)” refer to types 1 and 2, respectively, helper T lymphocyte (Th) mediated immune responses. Th2 responses include the allergy-associated IgE antibody class as well as elevated levels of IL-4 and IL-5 cytokines by Th2 lymphocytes. Th1 cells secrete IL-2, interferon-gamma (IFNγ) and tumor necrosis factor-beta (TNFβ) (the latter two of which are involved in macrophage activation and delayed-type hypersensitivity in response to antigen stimulation or infection with a pathogen).

Accordingly, Th2 associated responses can be suppressed, thereby reducing the risk of prolonged allergic inflammation and antigen-induced anaphylaxis. The enhancement of Th1 associated responses is of particular value in responding to intracellular infections because cellular immunity is enhanced by activated Th1 (IFNγ) cells. In addition, administration of polynucleotides helps stimulate production of CTL, further enhancing the immune response.

The method of the invention can be used to modulate or enhance the immune response both prophylactically and therapeutically. Thus, the invention provides a method of immunizing a subject as well as a method of immunotherapy.

The method of the invention comprises administering a composition comprising chitin to a subject prior to exposure to the substance. This priming is typically performed at least one hour prior to antigen administration or other exposure to a substance. The chitin micro-particles are preferably administered between about 6 hours and about 6 weeks prior to antigen administration or other exposure to a substance, and more preferably between about 1 day and about 4 weeks prior to antigen administration. Most preferably, the chitin micro-particles are administered between about 1 day and about 3 days prior to antigen administration. The antigen or other substance can be introduced by conventional immunization techniques, or by natural exposure.

Preferably, the substance is an antigen or a pathogen associated with an infectious disease, an allergen or a cancer. Examples of infectious disease include, but are not limited to, viral, bacterial, mycobacterial and parasitic diseases. Examples of allergens include, but are not limited to, plant pollens, dust mite proteins, animal dander, saliva and fungal spores. Examples of cancer-associated antigens include, but are not limited to, live or irradiated tumor cells, tumor cell extracts and protein subunits of tumor antigens. In some embodiments, the antigen is an environmental antigen. Examples of environmental antigens include, but are not limited to, respiratory syncytial virus (“RSV”), flu viruses and cold viruses.

The invention provides compositions that are useful for treating and preventing disease, such as allergy, cancer or infection. In one embodiment, the composition is a pharmaceutical composition. The composition is preferably an immunotherapeutic composition. The composition can comprise a therapeutically or prophylactically effective amount of chitin micro-particles of the invention, as described above. The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention.

In a preferred embodiment, the concentration of chitin micro-particles in a composition comprises about 1×10³ particles/mg up to 5×10⁹ particles/mg.

In another preferred embodiment, the chitin micro-particles are present in a concentration of about 1×10⁸ particles/mg to about 6×10⁸ particles/mg.

In another preferred embodiment, the chitin micro-particles are present at a concentration of about 1×10⁸ particles/mg to about 3×10⁸ particles/mg when the chitin micro-particle diameter is about 1 μm to about 10 μm.

In another preferred embodiment, the chitin micro-particles are present at a concentration of about 3×10⁸ particles/mg to about 6×10⁸ particles/mg when the chitin micro-particle diameter is about 1 μm to about 4 μm.

These concentrations can also vary depending on whether the adjuvant, chitin micro-particles, is to modulate the immune response to a Th1 type or whether it is to be used in combination with other antigens, vaccines etc. For example, the composition can fluffier comprise Mycobacterial antigens, MDP-59 or ragweed allergens. Other antigens include, peanut allergens, tree nut allergens, pollen allergens, house dust mite antigens, cockroach allergens, ovalbumin, mycobacterial heat-shock protein 65 and any components in the purified protein derivative (PPD).

In another preferred embodiment, the chitin micro-particles are used to treat immuno-suppressed individuals. Administration of the chitin micro-particle compositions comprises administering to such patients, e.g. HIV patients with a regimen of the chitin micro-particles to boost the patient's immune system and all the advantages that are associated with a stronger immune system.

Antigens

In those preferred embodiments wherein the chitin micro-particles are used as an adjuvant and an immune shifter to a Th1 response, different types of antigens can be included in the compositions. The antigen is selected so that the immune response, when elicited, will provide some level of therapeutic effect to the vaccinated individual, for example some level of effective protection against a disease agent. In those embodiments where it is intended that the immune response from a DNA vaccine be modified to enhance the Th1 character of the immune response, the antigen encoded by the DNA in the vaccine will be selected with this effect in mind.

In another preferred embodiment, the chitin micro-particles can be used to treat patients who are poor responders to a vaccine or other immunizing agent. For example, some patients are poor responders to Hepatitis B vaccine. The administration of the vaccine and chitin micro-particles can be used to provide a stronger immune response.

Examples of antigens that can be co-administered with the chitin micro-particles are limitless. Specific examples include tumor specific antigens. Tumor-specific antigens include, but are not limited to, any of the various MAGEs (melanoma associated antigen E), including MAGE 1, MAGE 2, MAGE 3 (HLA-A1 peptide), MAGE 4, etc.; any of the various tyrosinases (HLA-A2 peptide); mutant ras; mutant p53; and p97 melanoma antigen. Other tumor-specific antigens include the Ras peptide and p53 peptide associated with advanced cancers, the HPV 16/18 and E6/E7 antigens associated with cervical cancers, MUC1-KLH antigen associated with breast carcinoma, CEA (carcinoembryonic antigen) associated with colorectal cancer, gp100 or MARTI antigens associated with melanoma, and the PSA antigen associated with prostate cancer. The p53 gene sequence is known (see e.g., Harris et al. (1986) Mol. Cell. Biol. 6:4650 4656) and is deposited with GenBank under Accession No. M14694.

Suitable viral antigens include, but are not limited to, antigens obtained or derived from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV).

Antigens from the herpesvirus family can be used in the present invention, including antigens derived or obtained from herpes simplex virus (HSV) types 1 and 2, such as HSV-1 and HSV-2 glycoproteins gB, gD and gH; antigens from varicella zoster virus (VZV), Epstein-Barr virus (EBV) and cytomegalovirus (CMV) including CMV gB and g4; and antigens from other human herpesviruses such as HHV6 and HHV7. (See, e.g. Chee et al. (1990) Cytomegaloviruses (J. K. McDougall, ed., Springer-Verlag, pp. 125 169; McGeoch et al. (1988) J. Gen. Virol. 69:1531 1574; U.S. Pat. No. 5,171,568; Baer et al. (1984) Nature 310:207 211; and Davison et al. (1986) J. Gen. Virol. 67:1759 1816).

HIV antigens, such as the gp120 sequences for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); and Modrow et al. (1987) J. Virol. 61:570 578) and antigens derived from any of these isolates will find use in the present methods. Furthermore, the invention is equally applicable to other immunogenic moieties derived from any of the various HIV isolates, including any of the various envelope proteins such as gp160 and gp41, gag antigens such as p24gag and p55gag, as well as proteins derived from the pol, env, tat, vif rev, nef vpr, vpu and LTR regions of HIV.

Antigens from other viruses include, members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV_{IIB}, HIV_{SF2}, HIV_{LAV}, HIV_{LAI}, HIV_{MN}); HIV-1_{CM235}, HIV-1_{US4}; HIV-2, among others. See, e.g. Virology, 3rd Edition (W. K. Joklik ed. 1988); Fundamental Virology, 2nd Edition (B. N. Fields and D. M. Knipe, eds. 1991), for a description of these and other viruses.

Examples of bacterial and parasitic antigens include those obtained or derived from known causative agents responsible for diseases such as Diptheria, Pertussis, Tetanus, Tuberculosis, Bacterial or Fungal Pneumonia, Cholera, Typhoid, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borrelia, and Trichinosis. Still further antigens can be obtained or derived from unconventional viruses or virus-like agents such as the causative agents of kuru, Creutzfeldt-Jakob disease (CJD), scrapie, transmissible mink encephalopathy, and chronic wasting diseases, or from proteinaceous infectious particles such as prions that are associated with mad cow disease.

Examples of allergens that may find use in the present invention include, but are not limited to, allergens from pollens, animal dander, grasses, molds, dusts, antibiotics, stinging insect venoms, and a variety of environmental, drug and food allergens. Common tree allergens include pollens from cottonwood, popular, ash, birch, maple, oak, elm, hickory, and pecan trees; common plant allergens include those from rye, ragweed, English plantain, sorrel-dock and pigweed; plant contact allergens include those from poison oak, poison ivy and nettles; common grass allergens include Timothy, Johnson, Bermuda, fescue and bluegrass allergens; common allergens can also be obtained from molds or fungi such as Alternaria, Fusarium, Hormodendrum, Aspergillus, Micropolyspora, Mucor and thermophilic actinomycetes; penicillin and tetracycline are common antibiotic allergens; epidermal allergens can be obtained from house or organic dusts (typically fungal in origin), from insects such as house mites (Dermatphagoides pterosinyssis), or from animal sources such as feathers, and cat and dog dander; common food allergens include milk and cheese (diary), egg, wheat, nut (e.g., peanut), seafood (e.g., shellfish), pea, bean and gluten allergens; common drug allergens include local anesthetic and salicylate allergens; antibiotic allergens include penicillin and sulfonamide allergens; and common insect allergens include bee, wasp and ant venom, and cockroach calyx allergens. Particularly well characterized allergens include, but are not limited to, the major and cryptic epitopes of the Der p I allergen (Hoyne et al. (1994) Immunology 83190 195), bee venom phospholipase M (PLA) (Akdis et al. (1996) J. Clin. Invest, 98:1676 1683), birch pollen allergen Bet v 1 (Bauer et al. (1997) Clin. Exp. Immunol. 107:536 541), and the multi-epitopic recombinant grass allergen rKBG8.3 (Cao et al. (1997) Immunology 90:46 51). These and other suitable allergens are commercially available and/or can be readily prepared following known techniques.

In another preferred embodiment, the chitin micro-particles can be administered in a composition comprising one or more other adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); mineral gels; block copolymers; Avridine™ lipid-amine; SEAM62; adjuvants formed from bacterial cell wall components such as adjuvants including lipopolysaccharides (e.g., lipid A or monophosphoryl lipid A (MPL), Imoto et al. (1985) Tet. Lett. 26: 1545 1548), trehalose dimycolate (TDM), and cell wall skeleton (CWS); heat shock protein or derivatives thereof; adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, Staphylococcus aureus EDIN, and ADP-ribosylating bacterial toxin mutants such as CRM197, a non-toxic diphtheria toxin mutant (see, e.g., Bixler et al. (1989) Adv. Exp. Med. Biol. 251:175; and Constantino et al. (1992) Vaccine); saponin adjuvants such as Quil A (U.S. Pat. No. 5,057,540), or particles generated from saponins such as ISCOMs (immunostimulating complexes); chemokines and cytokines, such as interleukins (e.g., IL-1 IL-2, IL-4, IL-5, IL-6, IL-7, IL-8, IL-12, etc.), interferons (e.g., γ-interferon), macrophage colony stimulating factor (M-CSF), tumor necrosis factor (TNF), defensins 1 or 2, RANTES, MIP1-α and MIP-2, etc; muramyl peptides such as N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-s-n-glycero-3 hydroxyphosphoryloxy)-ethylamine (MTP-PE) etc; adjuvants derived from the CpG family of molecules, CpG dinucleotides and synthetic oligonucleotides which comprise CpG motifs (see, e.g., Krieg et al. Nature (1995) 374:546, Medzhitov et al. (1997) Curr. Opin. Immunol. 9:4 9, and Davis et al. J. Immunol. (1998) 160:870 876) and ATC C. limosum exoenzyme and synthetic adjuvants such as PCPP (Poly[di(carboxylatophenoxy)phosphazene) (Payne et al. Vaccines (1998) 16:92 98). Such adjuvants are commercially available from a number of distributors such as Accurate Chemicals; Ribi Immunechemicals, Hamilton, Mont.; GIBCO; Sigma, St. Louis, Mo.

Preferred adjuvants for use in the present compositions are those that are at least partially soluble in ethanol. A particularly preferred class of adjuvants for use herein are those classified as “saponins,” that is, adjuvants originating from saponin producing plants of the genera Quillaja, Saponaria, or Gypsophilia. Saponins are glycosidic natural plant products, composed of a ring structure (the aglycone) to which is attached one or more sugar chains. The aglycone can be asteroid, triteipenoid or a steroidalalkaloid and the number of sugar attached to the glycosidic bonds can vary greatly. The most common saponins used as pharmaceutical adjuvants are the triterpene glycosides extracted from the South American tree Quillaja saponaria and are referred to as Quil A (see e.g., U.S. Pat. Nos. 5,688,772; 5,057,540; and 4,432,969; and International Publication No. WO 88/09336, published 1 Dec. 1988), the active component of which is termed QS-21. Another preferred adjuvant is a muramyl dipeptide analog termed “GMTP-N-DPG” (N-acetylglucosaminyl-N-acetylmuramyl-L-alanyl-D-isoglutamyl-L-alanyl-dip-almitoylpropylamide). See Fast et al. (1997) Vaccine 15:1748 1752.

The adjuvant may be present in the instant compositions individually or in a combination of two or more adjuvants. In this regard, combined adjuvants may have an additive or a synergistic effect in promoting or shifting an immune response. A synergistic effect is one where the result achieved by combining two or more adjuvants is greater than one would expect than by merely adding the result achieved with each adjuvant when administered individually.

Administration and Dosage

In a preferred embodiment of the method, the chitin micro-particle compositions are administered via a systemic or mucosal route, or directly into a specific tissue, such as the liver, bone marrow, or into the tumor in the case of cancer therapy. Examples of systemic routes include, but are not limited to, intradermal, intramuscular, subcutaneous and intravenous administration. Examples of mucosal routes include, but are not limited to, intranasal, intravaginal, intrarectal, intratracheal and ophthalmic administration. Mucosal routes, particularly intranasal, intratracheal and ophthalmic, are preferred for protection against natural exposure to environmental pathogens such as RSV, flu viruses and cold viruses or to allergens such as grass and ragweed pollens and house dust mites. The local activation of innate immunity by the chitin micro-particles will enhance the protective effect against a subsequently encountered substance, such as an antigen, allergen or microbial agent.

Treatment includes prophylaxis and therapy. Prophylaxis or therapy can be accomplished by a single direct administration at a single time point or multiple time points. Administration can also be delivered to a single or to multiple sites.

The subject can be any vertebrate, but will preferably be a mammal. Mammals include human, bovine, equine, canine, feline, porcine, and ovine animals. If a mammal, the subject will preferably be a human, but may also be a domestic livestock, laboratory subject or pet animal.

The compositions of the present invention preferably contain a physiologically acceptable carrier. While any suitable carrier known to those of ordinary skill in the art may be employed in the inventive compositions, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the compositions of this invention. Suitable biodegradable microspheres are disclosed, for example, in U.S. Pat. Nos. 4,897,268 and 5,075,109. The compositions of the present invention may also contain a substance designed to protect the antigen from rapid catabolism, such as aluminum hydroxide or mineral oil.

In general, the inventive compositions maybe administered by injection (e.g., intradermal, intramuscular, intravenous or subcutaneous), intranasally (e.g., by aspiration) or orally. In certain embodiments, the compositions of the present invention are in a form suitable for delivery to the mucosal surfaces of the airways leading to or within the lungs. For example, the composition may be suspended in a liquid formulation for delivery to a patient in an aerosol form or by means of a nebulizer device similar to those currently employed in the treatment of asthma.

The preferred frequency of administration and effective dosage will vary both from individual to individual, and with the known antigen against which an immune response is to be raised, and may parallel those currently being used in immunization with the known antigen. In general, the amount of polypeptide immunostimulant present in a dose (or produced in situ by the polynucleotide in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 ml to about 2 ml.

In some preferred embodiments, the chitin micro-particles are administered from about 0.1-500 mg per kg of host. These amounts can be varied such as for example, amounts of about are 1-4 mg per kg for intraperitoneal or subcutaneous administration, or 1-500 mg per kg for oral administration.

The word “about,” when used in this application with reference to the amount of active component in a dose, contemplates a variance of up to 5% from the stated amount.

Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

Purification of Chitin Micro-Particles

We have established a method to prepare chitin micro-particles that induce macrophage activations, mediating bactericidal effects and Th1 adjuvant effects.

Brief procedure: 1. Crude chitin preparation (C9213) that was purchased from Sigma; 2. Washed with endotoxin-free saline; 3. Acid-soluble chitin was extracted by a mixture of chitin with cold 12 N hydrochloric acid (HCl) for 30 minutes; 4. Acid-soluble chitin was isolated by centrifugation at 1,500×g, 15 minutes, at 4° C.; 5. Acid-soluble chitin was neutralized at pH 7 with cold 10 N-sodium hydroxide (NaOH); 6. Chitin became insoluble and precipitated; 7. Insoluble chitin was washed with saline 5 times by centrifugation at 1,500×g for 15 minutes each at 4 C; 8. Following adjusting pH to 7.0, insoluble chitin was collected by centrifugation; 9. The pellets were lyophilized, and stored in desiccators.

Immediately before use: 10. Lyophilized chitin powder was suspended in saline at about 50 mg/ml; 11. The suspensions were sonicated on ice; 12.1-10 μm chitin particles were filtered through nylon mesh with 10-μm pores. The median distribution of particles was 4-5 μm. 1-4 μm chitin particles were further filtered through nylon mesh with 4-μm pores. 13. Filtered particles were collected and washed 4 times with saline by centrifugation at 1,500×g for 15 minutes each at 4° C.; 14. The concentrations were adjusted at 10 mg/ml or 16 mg/ml before use.

### Example 1

### Chitin Micro-Particles as an Adjuvant

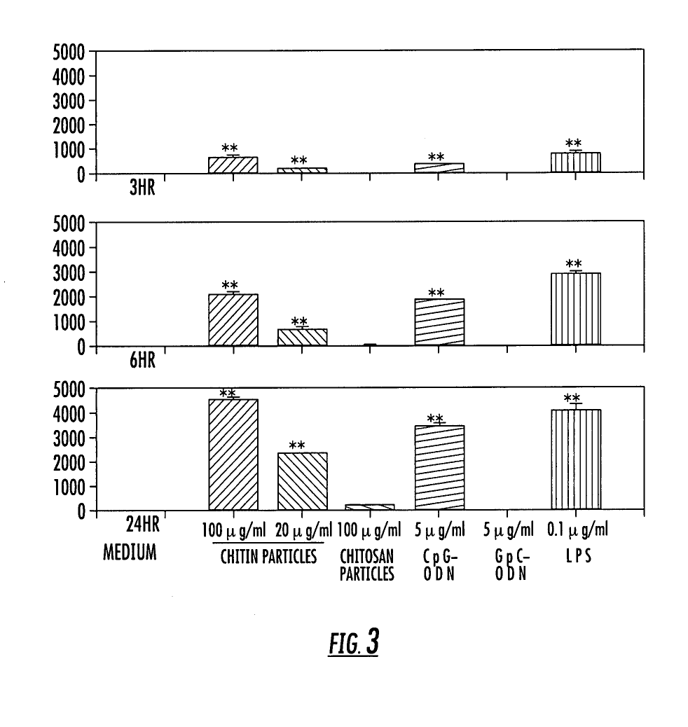

Biological activities measured in macrophage cultures: To measure the biological activities of chitin micro-particles prepared above, mouse macrophage RAW264.7 cells were stimulated with chitin particles at 20 or 100 μg/ml for 3, 6 and 24 hours. Levels of tumor necrosis factor-alpha (TNFα), a Th1 cytokine, in the supernatants were determined by a commercially available ELISA kit (PharMingen, San Diego, Calif.). As positive controls, bacterial DNA (CpG-ODN) at 5 μg/ml and bacterial endotoxin (LPS) at 0.1 μg/ml were used for the comparison study. As negative controls, medium alone (unstimulated control), chitosan micro-particles (de-acetylated chitin particles) at 100 μg/ml, and GpC-ODN at 5 μg/ml were used. As shown in FIG. 3, our newly prepared chitin particles induced TNFα producing activities at levels comparable to those by bacterial CpG and LPS.

Chitosan (de-acetylated chitin) micro-particles did not induce TNFα production, indicating no Th1 adjuvant activity. It is important that our purification method described above produces chitin micro-particles but not chitosan micro-particles.

Oral chitin protects from lethal challenges of Listeria monocytogenes: To determine whether our chitin micro-particles protect mice from lethal doses of Listeria monocytogenes, a single 8 mg oral dose chitin micro-particles was given selected mice. Normal Balb/c and C57Bl/6 are known to be sensitive and resistant to the infection, respectively; IL-10-knockout C57Bl/6 (IL-10^{-/-} mice are more resistant than C57Bl/6 (wild type, WT) mice; and, apolipoprotein E-knockout C57Bl/6 (apoE^{-/-}) mice, a hypercholesterolemic strain used for the model of atherosclerosis, were more sensitive to L. monocytogenes infection than C57Bl/6 (WT) mice.

As shown in Table 1, the treatment with chitin micro-particles protected all mice employed in this study from L. monocytogenes challenges up to 10×LD₅₀ (Table 1). L. monocytogenes was chosen for this study because this gram-positive intracellular organism has been used extensively in studies of cellular immune responses. In immunocompetent humans, it is typically associated with a mild food borne illness (Garifulin, O., Boyartchuk, V. (2005) Brief Funct Genomic Proteomic 4, 258-69). This organism is also potentially an agent of biowarfare.

TABLE 1

| | 10⁵ CFU | | 10⁶ CFU | | 10⁷ CFU | |
|---|---|---|---|---|---|---|
| | Saline | Chitin | Saline | Chitin | Saline | Chitin |
| Balb/c | 5/5 | 5/5 | 0/5 | 5/5* | 0/5 | 0/5 |
| WT (C57B1/6) | 5/5 | 5/5 | 2/5 | 5/5* | 0/5 | 2/5 |
| ApoE^{-/-} | 3/5 | 5/5 | 0/5 | 5/5* | 0/5 | 0/5 |
| IL-10^{-/-} | 5/5 | 5/5 | 4/5 | 5/5 | 0/5 | 4/5* |

The results from Table 1 show that oral chitin protects immunocompromised and control mice from lethal and sublethal challenges of Listeria monocytogenes. Groups of 5 female mice, 10-20 weeks old, were given 8 mg chitin particles orally. Two hours later, 10⁵ CFU, 10⁶ CFU, or 10⁷ CFU of Listeria monocytogenes (10403S serotype 1/2a) were administered intra-peritoneally. Mortality was recorded for 12 days. All live mice in all groups showed undetectable CFU levels (<10 CFU) in the spleen, lung and liver 12 days after the challenges with Listeria (data not shown). Results are for 5 mice for each mouse strain or genotype and each treatment group. Results shown were obtained in two separate experiments. Numbers are survivors at 12 days/total animals in each group. *The differences in survival data between chitin treatment group and saline control group were statistically significant (p<0.01); log rank test).

Local macrophage priming by ip administration of chitin particles: To kill intracellular bacteria such as L. monocytogenes, ROI and RNI (reactive oxygen and nitrogen intermediates) should be generated in the macrophages (MØ) (Shibata, Y., et al. (1998) J Immunol 161, 4283-8; Myers, J. T., T et al. (2003) J Immunol 171, 5447-53) (see FIG. 1). Myrvik et al (Myrvik, Q. N. et al. (1993) J Leukoc Biol 54, 439-43) and Shibata et al (Shibata, Y., et al (1998) J Immunol 161, 4283-8; Shibata, Y., et al (1997) Infect Immun 65, 1734-41; Shibata, Y., et al. (2001) Infect Immun 69, 6123-30) found that the administration of phagocytosable particles including chitin to experimental animals primes local macrophage non-specifically within 1-3 days to yield up to a 100-fold increase in their oxidative burst in vitro when elicited with phorbol myristate acetate (PMA).

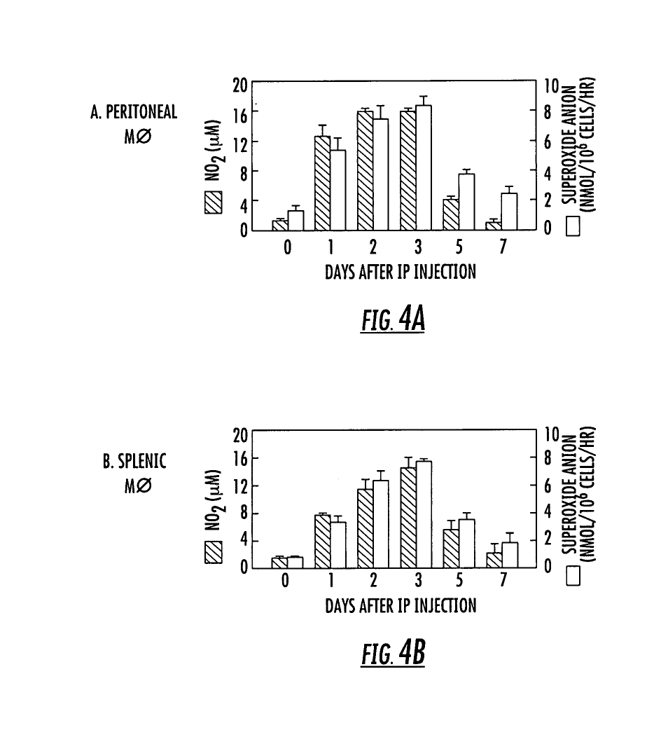

We examined whether intraperitoneal administration of our chitin micro-particles prime peritoneal macrophages and splenic macrophages. FIG. 4 shows that these macrophages released superoxide anion (ROS) as well as NO (RNS). C57Bl/6 mice received 1 mg chitin micro-particles ip. At indicated intervals, peritoneal and splenic macrophages were isolated. To measure NO release by Griess Reagent, macrophages were stimulated with LPS (1 μg/ml) for 24 hrs. To measure superoxide anion release by cytochrome c reduction, macrophages were stimulated with PMA (1 μM) for 1 hr. Mean±SD, n=3.

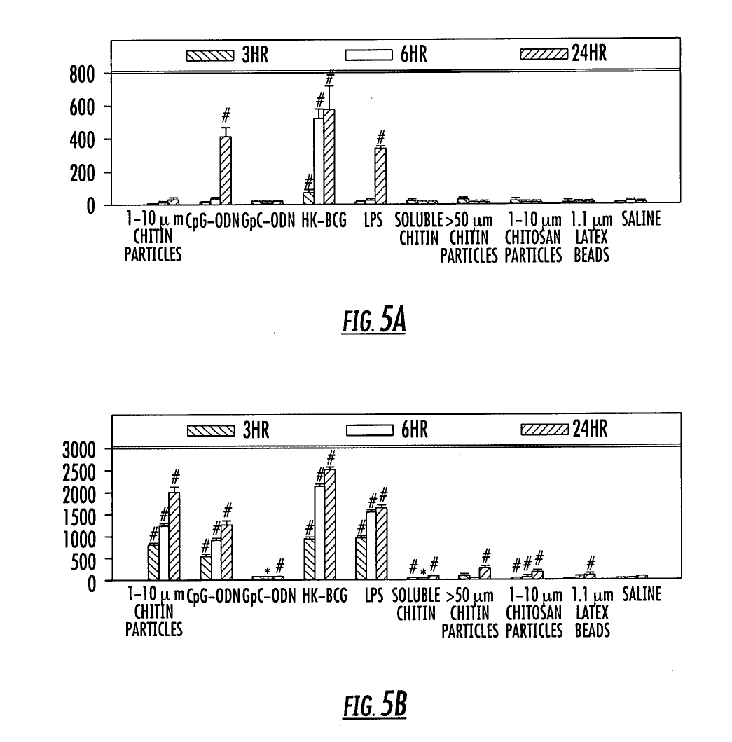

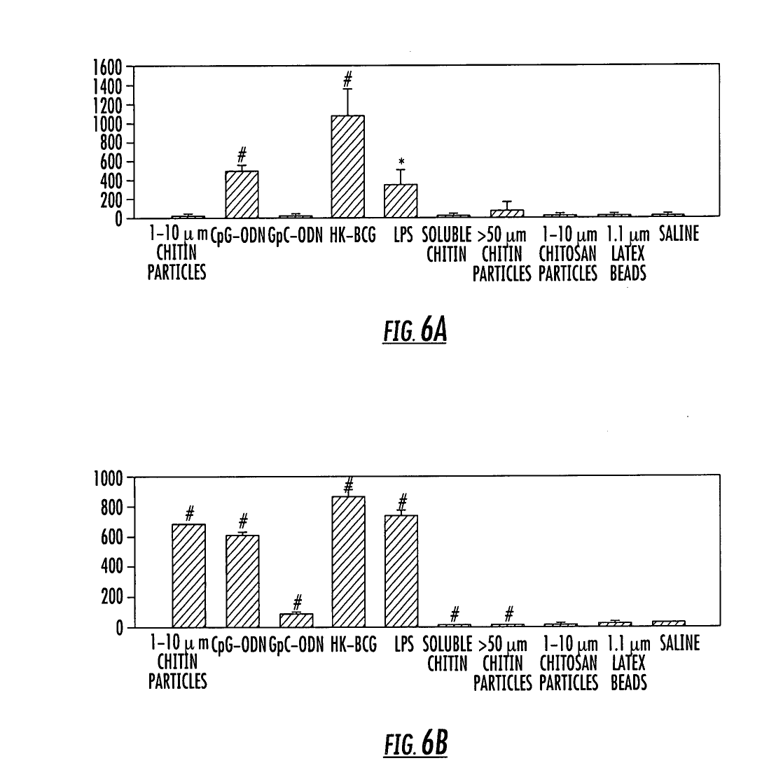

Chitin does not induce IL-10 production: As shown in FIG. 2, IL-10 inhibits Th1 adjuvant activities. An ideal Th1 adjuvant should not induce IL-10 production. We found that chitin micro-particles induce TNFα Th1 cytokine production but do not induce detectable IL-10 production. This finding is based on the experiments shown in FIGS. 5 and 6. In addition to TNFα production, we have determined that chitin micro-particles induce IL-12 and IL-18 as indicated previously (Shibata, Y., et al (1998) J Immunol 161, 4283-8).

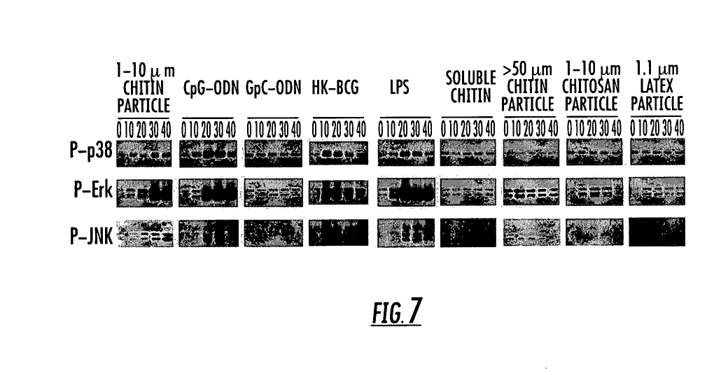

MAP kinases are phosphorylated by macrophage phagocytosing chitin particles but not chitosan or latex particles: As shown in FIG. 2, macrophages (MØ) phagocytose chitin micro-particles followed by Th1 cytokine production within 3-24 hrs. However, when macrophages phagocytose inert particles (latex beads, chitosan micro-particles), macrophages do not produce Th1 cytokines. It is known that macrophage activation by immunomodulators results in the phosphorylation of MAPK (p38, Erk1/2 and JNK) within 10-40 minutes. FIG. 7 shows that chitin particles induced phosphorylation of p38, Erk 1/2, and JNK (P-p38, P-Erk1/2 and P-JNK) in RAW264.7 macrophages at 10-40 minutes. The kinetics of chitin-induced phosphorylation were comparable to those observed for bacterial Th1 adjuvants (CpG-ODN and HK-BCG) or endotoxin (LPS). However, neither soluble chitin, >50 μm chitin particles, 1-10 μm chitosan nor 1.1 μm latex beads activated MAPK families.

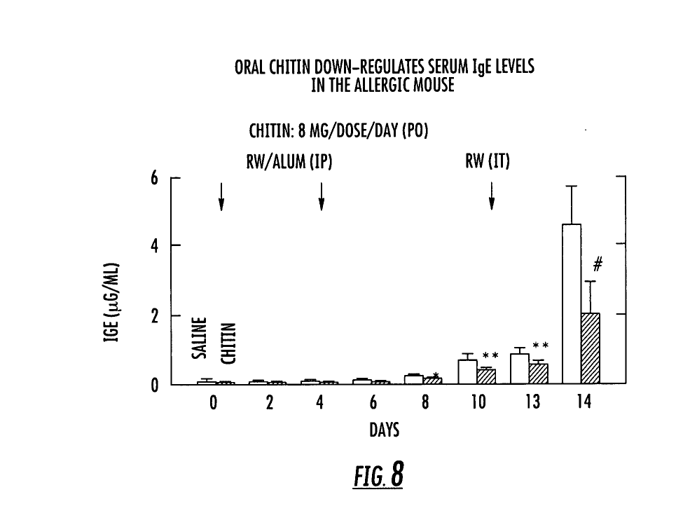

Oral chitin inhibits allergic responses: In a murine model of allergic airway disease, daily oral doses of 8 mg chitin increase Th1 responses with decreasing both serum IgE levels and lung eosinophil numbers by 60%. It should be noted that mice receiving chitin orally showed no distress but exhibited a slight weight gain. FIG. 8 shows a representative result of oral administration of chitin micro-particles inhibiting blood IgE levels in the mouse model of allergic asthma.

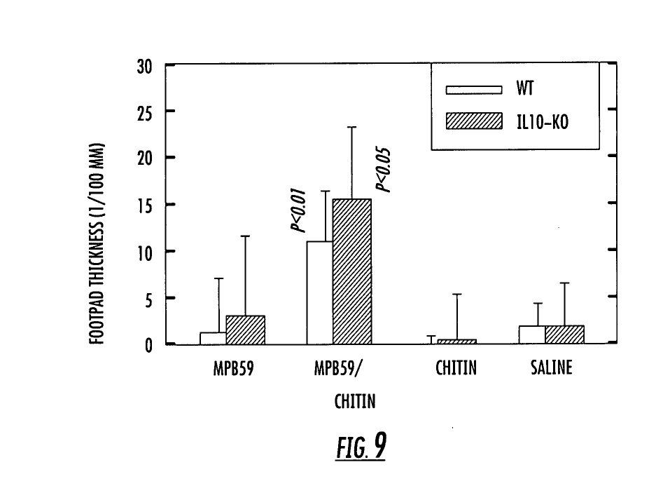

Chitin micro-particles are a Th1 adjuvant that induces antigen-specific delayed type hypersensitivity (DTH): We have determined that immunization of mice with soluble bacterial protein (antigen) mixed with chitin micro-particles establish the antigen-specific Th1 immunity. Our studies indicate that when C57Bl/6 (WT) mice and IL-10-knockout (IL-10-KO) mice are immunized with soluble MPB-59, a 30-kDa mycobacterial protective antigen, mixed with chitin micro-particles, chitin micro-particles effectively enhance the antigen-specific Th1 response (cell-mediated immunity), delayed type hypersensitivity (DTH). FIG. 9 shows the development of MPB-59-induced footpad delayed type hypersensitivity in C57Bl/6 (WT) and IL-10-knockout (IL-10-KO) mice co-immunized with MPB-59 and chitin micro-particles. Seven days after the final immunization, WT and IL-10-KO mice received 50 μg of MPB-59 solution in right footpads and 50 μl of saline in left footpads (control). After 48 hours, right footpad thickness minus left footpad thickness in each group of mice was obtained.

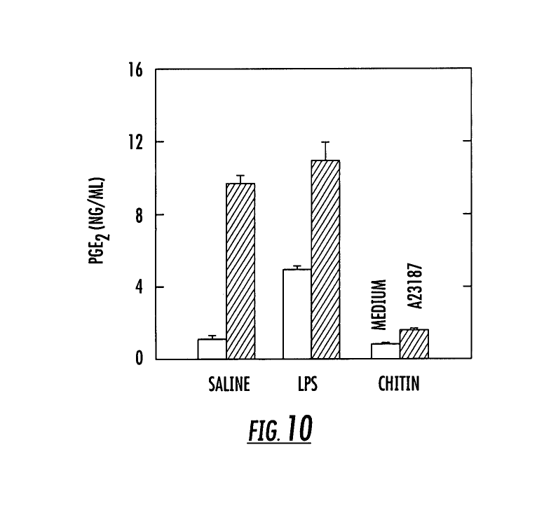

Intraperitoneal administration of chitin micro-particles inhibited PGE₂ release by peritoneal macrophages: As shown in FIG. 2, PGE₂ inhibits Th1 adjuvant activities. An ideal Th1 adjuvant should minimize PGE₂ production. PGE₂ is known to be synthesized during infections and inflammation by activation of COX-2 (cyclooxygenase-2) in macrophages. We have determined that administration of chitin micro-particles results in the modification of COX-2 in local MØ. The modified COX-2 loses catalytic activity for the synthesis of PGE₂. FIG. 10 indicates that when C57Bl/6 mice are given 1 mg chitin micro-particles intraperitoneally, the capacity for PGE₂ biosynthesis by peritoneal MØ ex vivo is significantly reduced. Peritoneal macrophages were isolated 24 hrs after the stimulation. Macrophage suspension (10⁶/ml) was stimulated with 1 μM calcium ionophore 23187 (black bars) or medium (gray bars), for 2 hrs to determine PGE₂ release. PGE₂ was assayed by ELISA.

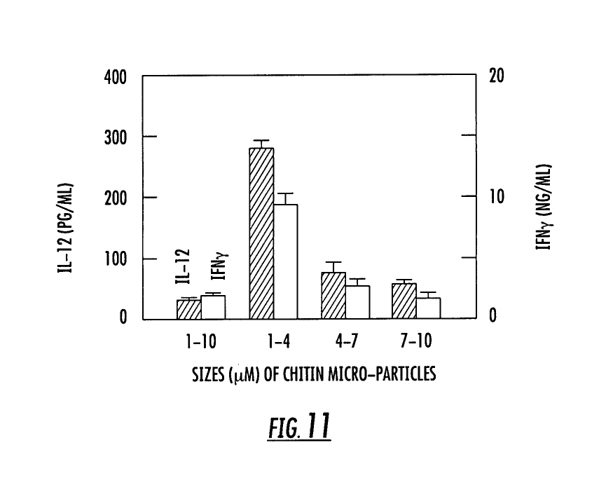

New 1-4 μm particles of chitin (1-4 μm chitin) preparations: As shown in FIGS. 1 and 2, chitin micro-particles induce Th1 cytokines including IL-12 that activates NK cells to produce IFNγ within 24 hours. The inductions of IL-12 and IFNγ production are the key Th1 adjuvant activities. To prepare chitin micro-particles expressing better Th1 adjuvant activities, 1-10 μm chitin micro-particles were further fractionated at 1-4 μm, 4-7 μm and 7-10 μm sizes through nylon meshes. The biological activities were determined by adding each fraction to spleen cell cultures at 20 μg/ml. As shown in FIG. 11, the 1-4 μm fraction had the highest activity with respect to the IL-12 and IFNγ productions that were at least 5-fold greater than for the untreated 1-10 μm chitin preparation. Since 1-4 μm chitin contained about twice as many particle numbers as 1-10 μm chitin in equal masses (2.5×10⁸ particles/mg for 1-10 μm chitin), these results suggest that the chitin effects may be determined both by the size and number of internalized particles.

We have established the chitin micro-particle preparation at 1-10 μm sizes that can be scaled up to produce commercial levels of the product. Oral administration of our product prevented several strains of mice (Balb/c, C57Bl/6, IL-10-KO, apoE-KO), models of immunocompetent and immunocompromised human individuals, from lethal doses of Listeria monocytogenes infections (Table 1). Oral administration of chitin micro-particles inhibits allergic responses including blood IgE levels in the mouse allergic model (FIG. 8). Intraperitoneal administration of chitin micro-particles enhances Th1 responses against co-injected soluble protein antigens in the mouse model (FIG. 9). Chitin micro-particles produce the following immunoprotective responses: (a) Chitin micro-particles induce Th1 cytokine (TNFα, IL-12 and IL-18) but not IL-10 Th2 cytokine. IL-10, which is induced by bacterial components, is known to inhibit bactericidal activity and Th1 adjuvant activities (FIGS. 3, 5, 6 and 11); (b) Chitin micro-particles induce macrophages to produce reactive oxygen and nitrogen intermediates that directly attack intracellular bacteria (FIG. 4); (c) Chitin micro-particles activate mitogen-activating protein kinases (MAPK), whereas chitosan micro-particles or latex beads do not activate MAPK (FIG. 7). MAPK activation is essential to mediate macrophage bactericidal functions and Th1 adjuvant activities; (d) Chitin micro-particles inhibit PGE₂ biosynthesis (FIG. 10). PGE₂ inhibits Th1 cytokine production, but enhances Th2 cytokine production.

Chitin micro-particles at 1-10 μm have been tested above for their Th1 adjuvant activities. We have found, in addition, that chitin micro-particles at 1-4 μm sizes provide better Th1 adjuvant activities. The chitin micro-particles that we have established can be used to induce protective immunity against various inflammatory diseases including listeriosis and asthma in immunocompromised populations.

### Example 2

### Chitin Particles Inhibit CpG-ODN-Induced IL-10 Production But Do Not Inhibit IL-10 mRNA Synthesis

Untreated RAW264.7 macrophages produced 1,106 pg/ml IL-10 when stimulated with CpG-ODN (Table 2). CpG-ODN-stimulated macrophages were treated with chitin particles at selected time points before or after CpG-ODN stimulation. The results (Table 2) showed that chitin micro-particles inhibited significantly IL-10 production by 9 hours post-CpG-ODN stimulation.

TABLE 2
Chitin particles inhibit CpG-ODN-induced IL-10 production by RAW
264.7 macrophages.

Stimulation (37° C., 24 h)

| | | |
|---|---|---|
| Time point of | CpG-ODN | Saline^{b} |

| | |
|---|---|
| chitin addition^{a} | IL-10 pg/ml (% Production) |

| | | |
|---|---|---|
| No particle | 1,106 ± 31 (100) | 29 ± 14 |
| −1 h | 181 ± 9^{#} (16) | 18 ± 17 |
| 0 h | 187 ± 19^{#} (17) | 18 ± 8 |
| 1 h | 245 ± 44^{#} (22) | 17 ± 4 |
| 3 h | 314 ± 5^{#} (28) | 38 ± 14 |
| 6 h | 459 ± 5^{#} (42) | <15^{c} |
| 9 h | 883 ± 5^{#} (80) | 32 ± 15 |
| 12 h | 991 ± 5 (90) | 16 ± 5 |

^{a}The time point when CpG-ODN was added to culture was considered as 0 h. Chitin microparticles were added to culture at the indicated time points. Macrophage stimulation was terminated at 24 h.
^{b}Mean ± SD, n = 3. ( ), % Production compared to cells stimulated CpG-ODN alone.
^{#}p < 0.01, compared to cells stimulated CpG-ODN alone. The data shown are representative of three independent experiments.
^{c}15 pg/ml of IL-10 was the lower limit of detection.

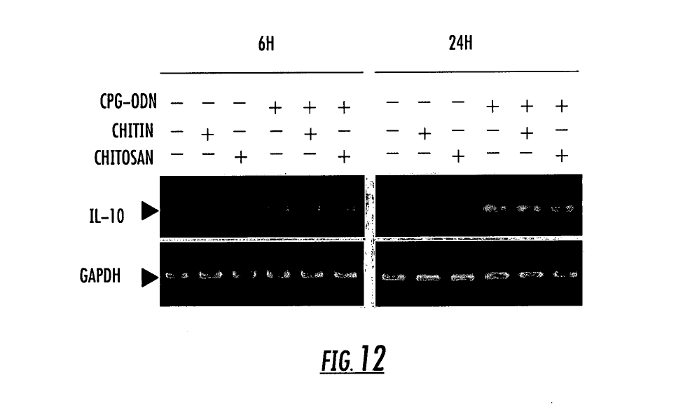

FIG. 12 are blots showing the effects of chitin particles on the levels of CpG-ODN-induced IL-10 mRNA. RAW264.7 MØ (5×10⁵/ml) were stimulated with CpG-ODN alone or combination with chitin particles or chitosan particles at 37° C. for 6 and 24 h. Total RNA was extracted from the cells with Trizol reagent (Invitrogen, Carlsbad, Calif.) according to the manufacturer's instructions. IL-10 mRNA expression was examined by RT-PCR. Reverse transcription of mRNA was achieved by SuperScript™ First-Strand Synthesis System for RT-PCR (Invitrogen) with oligo-(dT) primer according to the manufacturer's instructions. PCR primers used were: IL-10 (forward: 5′-GGT TGC CAA GCC TTA TCG GA-3′ (SEQ ID NO: 1), reverse: 5′-ACC TGC TCC ACT GCC TTG CT-3′ (SEQ ID NO: 2)) and GAPDH (forward: 5′-TTC ACC ACC ATG GAG AAG GC-3′ (SEQ ID NO: 3), reverse: 5′-GGC ATG GAC TGT GGT CAT GA-3′ (SEQ ID NO: 4)). Fifteen μl of PCR products was electrophoresed on 2% agarose gel. After ethidium bromide staining, PCR products were visualized by UV illumination.

As shown in FIG. 12, chitin micro-particles did not induce detectable IL-10 mRNA, whereas CpG-ODN induced IL-10 mRNA 6 and 24 hours after the stimulation. However, chitin micro-particles did not alter CpG-ODN-induced IL-10 mRNA levels at 6 h and 24 h after the stimulation with a mixture of chitin micro-particles and ODN-CpG (FIG. 12). These results indicate that chitin particles inhibit IL-10 production at a post-transcriptional level.

### Other Embodiments

It is to be understood that while the invention has been described in conjunction with the detailed description thereof, the foregoing description is intended to illustrate and not limit the scope of the invention. Other aspects, advantages, and modifications are within the scope of the following claims.

All references cited herein, are incorporated herein by reference.

What is claimed is:

1. An adjuvant composition comprising at least one chitin micro-particle wherein the micro-particles are about 0.01 μm up to 20 μm in diameter.

2. The adjuvant composition of claim 1, wherein the chitin micro-particles are between about 1 μm to 10 μm in diameter.

3. The adjuvant composition of claim 1, wherein the chitin micro-particles are between about 1 μm to 4 μm.

4. The adjuvant composition of claim 1, wherein the chitin micro-particles are suspended in a pharmaceutical composition in an amount effective in decreasing T-helper type 2 cell activity and increase T-helper type 1 cell activity.

5. The adjuvant composition of claim 1, wherein the chitin micro-particles are present in a concentration of about 1×10³ particles/mg up to 5×10⁹ particles/mg.

6. The adjuvant composition of claim 5, wherein the chitin micro-particles are present in a concentration of about 1×10⁸ particles/mg to about 6×10⁸ particles/mg.

7. The adjuvant composition of claim 5, wherein the chitin micro-particles are present at a concentration of about 1×10⁸ particles/mg to about 3×10⁸ particles/mg when the chitin micro-particle diameter is about 1 μm to about 10 μm.

8. The adjuvant composition of claim 5, wherein the chitin micro-particles are present at a concentration of about 3×10⁸ particles/mg to about 6×10⁸ particles/mg when the chitin micro-particle diameter is about 1 μm to about 4 μm.

9. The adjuvant composition of claim 1, wherein the composition further comprises Mycobacterial antigens, MDP-59, ragweed allergens, peanut allergens, tree nut allergens, pollen allergens, house dust mite antigens, cockroach allergens, ovalbumin, mycobacterial heat-shock protein 65, antigens, and purified protein derivative (PPD) and derivatives thereof.

10. The adjuvant composition of claim 9, wherein the antigens comprise vaccines, tumor antigens, viral antigens, bacterial antigens, or peptides.

11. A method of preparing a chitin micro-particle comprising:
washing chitin crude sources with endotoxin free saline;
mixing the washed chitin with acid;
extracting and isolating acid soluble chitin;
neutralizing and precipitating soluble chitin;
washing water-insoluble chitin;
lyophilizing water-insoluble chitin;
re-suspending water-insoluble chitin in endotoxin free saline;
filtrating chitin particles through micro-pores;
preparing a 1-10 μm chitin micro-particle.

12. The method of claim 11, wherein a chitin-micro particle size is about 1 to 4 μm.

13. The method of claim 11, wherein the chitin micro-particles in saline are stable for at least one year at 4° C.

14. A method of treating and/or preventing an inflammatory disease comprising: administering to a patient a therapeutically effective dose of chitin micro-particles wherein the chitin micro-particles are between about 1 μm to 4 μm.

15. The method of claim 14, wherein the chitin micro-particles are administered at an amount comprising about 0.1-500 mg per kg of the patient.

16. The method of claim 14, wherein the chitin micro-particles are administered at an amount comprising about 0.1-10 mg per kg of the patient intraperitoneally or subcutaneously.

17. The method of claim 14, wherein the chitin micro-particles are administered at an amount comprising about 0.1-1 g per kg of the patient when administered orally.

18. The method of claim 14, wherein the chitin micro-particles are suspended in a pharmaceutical composition in an amount effective in decreasing T-helper type 2 cell activity and increase T-helper type 1 cell activity.

19. The method of claim 14, wherein the route of administration is oral, intra-peritoneal, intra-venous and/or under a patient's skin.

20. The method of claim 14, wherein administration of chitin micro-particles is therapeutically effective in down-regulating Th2-mediated diseases including allergic asthma, food allergy and allergic dermatitis.

21. The method of claim 14, wherein administration of chitin micro-particles is therapeutically effective in up-regulating Th1-host defenses against intracellular infections.

22. A method of modulating an immune response, comprising: administering to an animal an effective dose of chitin particles wherein the chitin micro-particles are between about 1 μm to 4 μm in diameter.

23. The method of claim 21, wherein the chitin micro-particles are suspended in a pharmaceutical composition in an amount effective in decreasing T-helper type 2 cell activity and increase T-helper type 1 cell activity.

24. The method of claim 21, wherein the route of administration is oral, intra-peritoneal, intra-venous and/or under a patient's skin.
